(19) United States
(12) Patent Application Publication
(10) Pub. No.: US 2020/0330053 A1
(43) Pub. Date: Oct. 22, 2020

(54) RADIOGRAPHIC IMAGING APPARATUS

(71) Applicant: Shimadzu Corporation, Kyoto (JP)

(72) Inventors: Dai HIROSE, Kyoto (JP); Koki YOSHIDA, Kyoto (JP); Daisuke MURAKAMI, Kyoto (JP)

(21) Appl. No.: 16/644,815

(22) PCT Filed: Nov. 15, 2017
(86) PCT No.: PCT/JP2017/041022

§ 371 Date: Mar. 5, 2020

(51) Int. Cl. A61B 6/04; A61B 6/00

(52) U.S. Cl. CPC A61B 6/04; A61B 6/462

(57) ABSTRACT

A radiographic imaging apparatus includes: a plurality of first operation portions configured to move the moving member in a selected moving direction by being operated to select a moving direction of the moving member; a second operation portion for preparing a movement of the moving member to a selected final movement position by being operated to select the second operation portion; a third operation portion for initiating the movement of the moving member to the final movement position by being operated after the second operation portion is operated; a lamp built in the first operation portion; a moving direction calculation unit for calculating the moving direction of the moving member to the final movement position from a current position and the selected final movement position of the moving member; and a display unit for displaying the lamp built in the first operation portion corresponding to the moving direction of the moving member to the final movement position by blinking the lamp.

### TECHNICAL FIELD

The present invention relates to a radiographic imaging apparatus equipped with a moving member movable in a plurality of directions for performing radiographic imaging and an operation panel for operating movements of the moving member.

### BACKGROUND OF THE INVENTION

An X-ray vascular fluoroscopic imaging apparatus as such a radiographic imaging apparatus is provided with an examination table and an X-ray imaging unit. The examination table is provided with a top board for placing a subject thereon and a top board rocking mechanism that moves the top board along the X-, Y-, and Z-directions and tilts or rolls the top board. The X-ray imaging unit is provided with an X-ray irradiation unit including an X-ray tube and a collimator, a radiation detector such as a flat panel detector for detecting radiation emitted from the X-ray irradiation unit and transmitted through the subject placed on the top board, a C-shaped arm that supports the X-ray irradiation unit and the radiation detector in an opposed state, and a C-shaped arm moving mechanism that supports the C-shaped arm in a slidable and rotatable manner (see Patent Document 1). Further, the X-ray fluoroscopic imaging apparatus is provided with an input unit for operating a top board so as to take various imaging postures (see Patent Document 2).

### PRIOR ART DOCUMENT

### Patent Document

Patent Document 1: Japanese Unexamined Patent Application Publication No. 2017-118910
Patent Document 2: Japanese Unexamined Patent Application Publication No. 2015-223245

### SUMMARY OF THE INVENTION

### Problems to be Solved by the Invention

In such an X-ray imaging apparatus, an operation panel is used to move the top board. For example, when horizontally moving the top board in the longitudinal direction, a switch corresponding to the horizontal movement in the longitudinal direction is pressed and the pressing of the switch is released when the top board has horizontally been moved and reached a predetermined position.

On the other hand, as the moving operation of such a top board, an automatic operation can also be selected. The automatic operation is an operation of automatically moving the top board to the origin position of the top board or a preset position by pressing a switch corresponding to the automatic operation.

When performing this automatic operation, the movement of the top board is initiated by selecting and pressing either a switch for instructing the movement to the origin position or a switch for instructing the movement to the preset position and then pressing a start switch. When the top board has reached the origin position of the top board or the preset position, the movement of the top board is stopped.

When moving the top board by the automatic operation as described above, it is possible to automatically move the top board without specifying the moving direction of the top board. However, the operator cannot predict the moving direction of the top board until the top board initiates the movement, so there is a risk that the operator collides with the top board. This phenomenon occurs not only in the top board but also in the X-ray imaging unit as well.

The present invention has been made to solve the aforementioned problems, and an object of the present invention is to provide a radiographic imaging apparatus capable of avoiding the risk of an operator colliding with a moving member by clearly indicating a moving direction of the moving member by a lamp.

### Means for Solving the Problem

According to the invention as recited in claim 1, a radiographic imaging apparatus equipped with a moving member movable in a plurality of directions for performing radiographic imaging and an operation panel for operating movements of the moving member, includes:

a plurality of first operation portions provided on the operation panel to move the moving member in a selected moving direction by being operated to select a moving direction of the moving member, the plurality of first operation portions being provided corresponding to moving directions of the moving member;

a second operation portion provided on the operation panel to prepare a movement of the moving member to a selected final movement position when the final movement position is selected by being operated;

a third operation portion arranged on the operation panel to initiate the movement of the moving member to the final movement position by being operated after the second operation portion is operated;

a lamp built in each of the plurality of first operation portions;

a moving direction calculation unit configured to calculate a moving direction of the moving member to the final movement position from a current position and the selected final movement position of the moving member; and

a moving direction display unit configured to turn on the lamp built in the first operation portion corresponding to the moving direction of the moving member to the final movement position calculated by the moving direction calculation unit when the second operation portion is operated.

According to the invention as recited in claim 2, in the invention as recited in claim 1, the moving direction display unit causes the lamp built in the first operation portion to blink.

According to the invention as recited in claim 3, in the invention as recited in claim 1 or 2, the moving member is a top board of an examination table movable in the plurality of directions with a subject placed thereon.

According to the invention as recited in claim 4, in the invention as recited in claim 1 or 2, the moving member is a support arm that supports a radiation irradiation unit and a radiation detection unit to move the radiation irradiation unit and the radiation detection unit in the plurality of directions.

### Effects of the Invention

According to the invention as recited in claim 1, the lamp built in the first operation portion corresponding to the moving direction of the moving member to the final movement position is turned on, so that an operator can recognize the moving direction of the moving member. For this reason, the risk of the operator colliding with the moving member can be avoided.

According to the invention as recited in claim 2, since the lamp is turned on, the moving direction of the moving member can be more clearly recognized.

According to the invention as recited in claim 3, the moving direction of the top board can be recognized, and therefore the risk of the operator colliding with the top board can be avoided.

According to the invention as recited in claim 4, since the moving direction of the support arm can be recognized, and therefore the risk of the operator colliding with the support arm can be avoided.

### BRIEF DESCRIPTION OF THE DRAWINGS

FIG. 1 is a perspective view of an X-ray vascular fluoroscopic imaging apparatus as the radiographic imaging apparatus according to the present invention, as viewed from the front side.

FIG. 2 is a perspective view of the X-ray vascular fluoroscopic imaging apparatus as the radiographic imaging apparatus according to the present invention, as viewed from the side.

FIG. 3 is a perspective view of the X-ray vascular fluoroscopic imaging apparatus as the radiographic imaging apparatus according to the present invention, as viewed obliquely from the rear side.

FIG. 4 is a perspective view schematically showing a top board moving mechanism of an examination table 1.

FIG. 5 is a perspective view of an operation panel 40.

FIG. 6 is a block diagram showing the main control system of an X-ray vascular fluoroscopic imaging apparatus.

### EMBODIMENTS FOR CARRYING OUT THE INVENTION

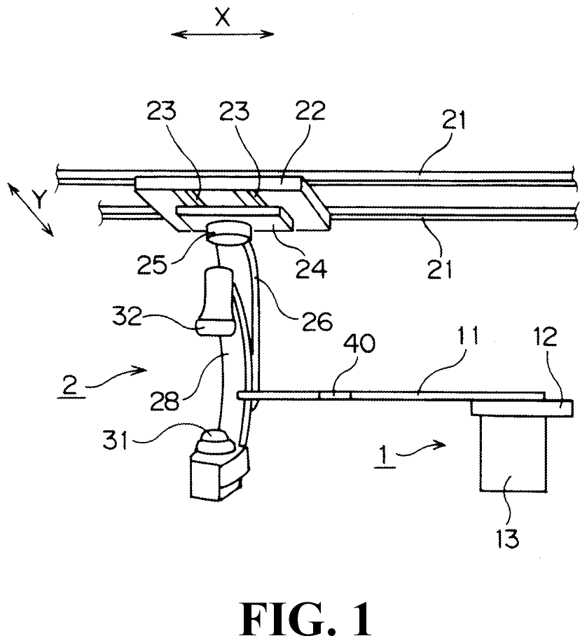

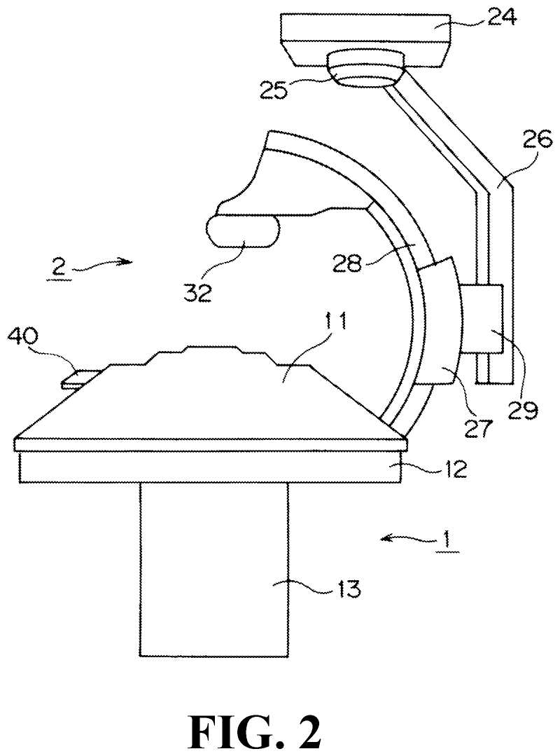

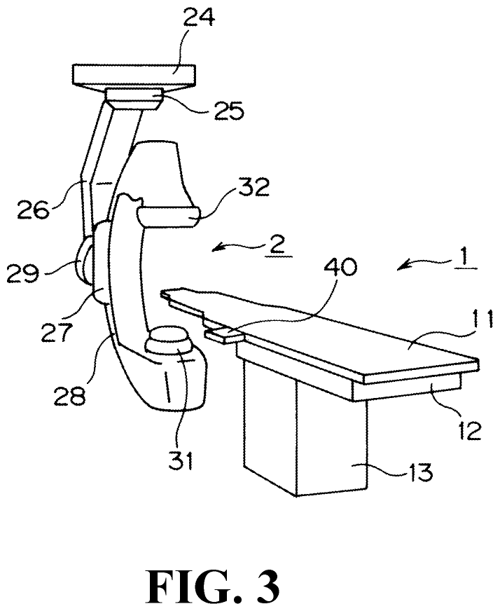

Hereinafter, embodiments of the present invention will be described with reference to the drawings. FIG. 1 is a perspective view of an X-ray vascular fluoroscopic imaging apparatus as the radiographic imaging apparatus according to the present invention, as viewed from the front side. FIG. 2 is a perspective view of the X-ray vascular fluoroscopic imaging apparatus as the radiographic imaging apparatus according to the present invention, as viewed from the side. FIG. 3 is a perspective view of the X-ray vascular fluoroscopic imaging apparatus as the radiographic imaging apparatus according to the present invention, as viewed obliquely from the rear side. Note that FIG. 1 and FIG. 2 show the state in which the C-shaped arm 28 is slid in the oblique direction and FIG. 3 shows the state in which the C-shaped arm 28 is slid in the sagittal direction.

The X-ray vascular fluoroscopic imaging apparatus as the radiographic imaging apparatus according to the present invention is provided with an examination table 1 and an X-ray imaging unit 2. The top board 11 of the examination table 1 and the C-shaped arm 28 of the X-ray imaging unit 2 each constitute the moving member according to the present invention that moves in a plurality of directions for performing X-ray imaging. Note that in the following description, the description will be directed to the case in which the movement of the top board 11 as a moving member is performed by an operation panel 40.

The X-ray imaging unit 2 is provided with an X-ray irradiation unit 31 as the radiation irradiation unit according to the present invention including an X-ray tube and a collimator, and a flat panel detector 32 as a radiation detector according to the present invention for detecting X-rays irradiated from the X-ray irradiation unit 31 and transmitted through a subject on the examination table 1. The X-ray imaging unit 2 is provided with a generally C-shaped arm 28 having an arc-shaped guide and supporting the X-ray irradiation unit 31 and the flat panel detector 32 in a faced manner, a sliding portion 27 slidably supporting the C-shaped arm 28 by being engaged with the guide of the C-shaped arm 28, a rotating portion 29 rotatably supporting the sliding portion 27, and a support portion 26 supporting the rotating portion 29.

The support portion 26 is supported by the first moving member 24 via a rotation member 25 that rotates about an axis extending in a vertical direction. The first moving member 24 is movable in the Y-direction shown in FIG. 1 along a pair of rails 23 attached on the lower surface of the second moving member 22. The first moving member 24 is movable in the X-direction shown in FIG. 1 along a pair of rails 21 attached to the ceiling of the examination room.

Therefore, the C-shaped arm 28 is movable between the position shown in FIG. 1 and FIG. 2 and the position shown in FIG. 3 by moving in the X-direction and the Y-direction together with the X-ray irradiation unit 31 and the flat panel detector 32 and rotating about the vertical axis direction, and is rotatable about the rotating portion 29 and slidable to the sliding portion 27.

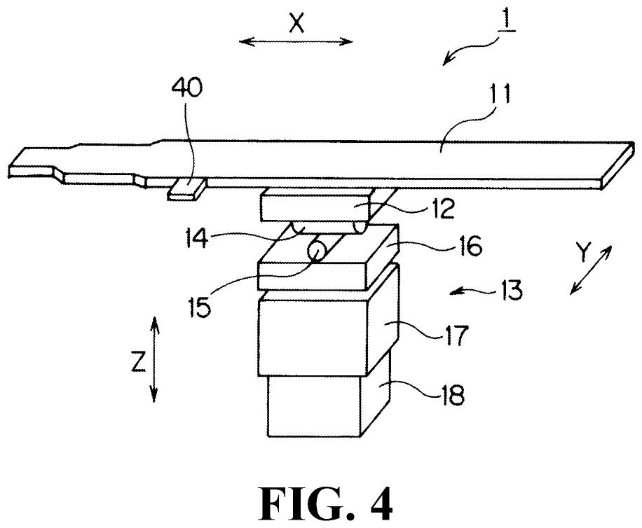

FIG. 4 is a perspective view schematically showing a top board moving mechanism in the examination table 1. In this figure, the inner structure of the base 13 shown in FIG. 1 to FIG. 3 is shown.

As shown in FIG. 1 to FIG. 4, the examination table 1 is composed of the top board 11 for placing a subject thereon, an operation panel 40 attached to the top board 11, a flame 12, and a base 13.

The top board 11 is supported in a movable manner with respect to the flame 12 in the longitudinal direction of the top board 11 (in the X-direction shown in FIG. 4 when the top board 11 is arranged in the horizontal direction). The flame 12 is swingable about the shaft 14 extending in the short direction (in the Y-direction shown in FIG. 4 when the top board 11 is arranged in the horizontal direction) and also swingable about the shaft 15 extending in the longitudinal direction. With this, the top board 11 can perform rolling that swings about the shaft 14 and tilting that swings about the shaft 15 together with the flame 12.

The shaft 14 and the shaft 15 are supported by a movable base 16. The movable base 16 is movable relative to a lifting base 17 in the short direction of the top board 11. The lifting base 17 is liftable in the vertical direction (in the Z-direction shown in FIG. 4) with respect to a support base 18. As described above, the top board 11 is movably supported in the longitudinal direction of the top board 11 with respect to the flame 12. For this reason, the top board 11 is movable in the longitudinal direction and short direction thereof, and is also liftable.

The operation panel 40 attached to the top board 11 is also referred to as a console, and is used to perform the aforementioned operations for moving the top board 11 in the longitudinal direction, the short direction, and the vertical direction and for rolling and tilting the top board 11.

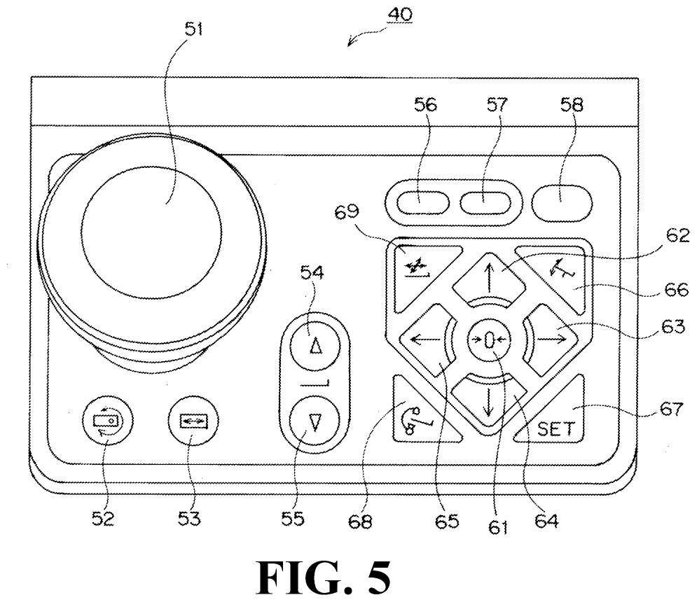

FIG. 5 is a perspective view of the operation panel 40.

This operation panel 40 is provided with a lever 51 for manually moving the top board 11 in an unlocked state, a lock release button 52 for releasing the locking of pivotable movements of the top board 11, and a lock release button 53 for releasing the locking of longitudinal movements of the top board 11. The operation panel 40 is also provided with an UP-button 54 for raising the top board 11 and a DOWN-button 55 for lowering the top board 11. The operation panel 40 is also provided with a first position memory move button 56 for automatically moving the top board 11 to a preset first position as a final movement position, a second position memory move button 57 for automatically moving the top board 11 to a preset second position as a final movement position, and an origin returning button 61 for automatically moving the top board 11 to an origin position as a final movement position. The operation panel 40 is also provided with a long/short select button 69 for selecting the movement of the top board 11 in the longitudinal direction or the movement of the top board 11 in the short direction, and a roll/tilt selection button 66 for selecting the rolling or tiling of the top board 11. The operation panel 40 is also provided with a first button 62 and a second button 64 for moving the top board 11 in the longitudinal direction after the long/short select button 69 is pressed and for tilting the top board 11 after the roll/tilt selection button 66 is pressed, and a third button 63 and a fourth button 65 for moving the top board 11 in the short direction after the long/short select button 69 is pressed and for rolling the top board 11 after the roll/tilt selection button 66 is pressed. The operation panel 40 is also provided with a cancel button 58 for canceling the operation previously performed, and an operation button 68 for tilting the top board 11 while maintaining the height of the end of the top board 11. Further, the operation panel 40 is provided with a movement start button 67 for initiating the movement of the top board 11 to the first position, the second position, or the origin position by being pressed after the first position memory move button 56, the second position memory move button 57 or the origin returning button 61 is pressed.

Note that the UP-button 54, the DOWN-button 55, the first button 62, the second button 64, the third button 63, and the fourth button 65 described above constitute a first operation portion according to the present invention. Also note that the first position memory move button 56, the second position memory move button 57, and the origin returning button 61 described above constitute the second operation portion according to the present invention. Further note that the movement start button 67 described above constitutes a third operation portion according to the present invention. A lamp is arranged in the UP-button 54, the DOWN-button 55, the first button 62, the second button 64, the third button 63, and the fourth button 65 constituting the first operation portion, and in the long/short select button 69 and the roll/tilt selection button 66. Note that a lamp may be arranged in other buttons.

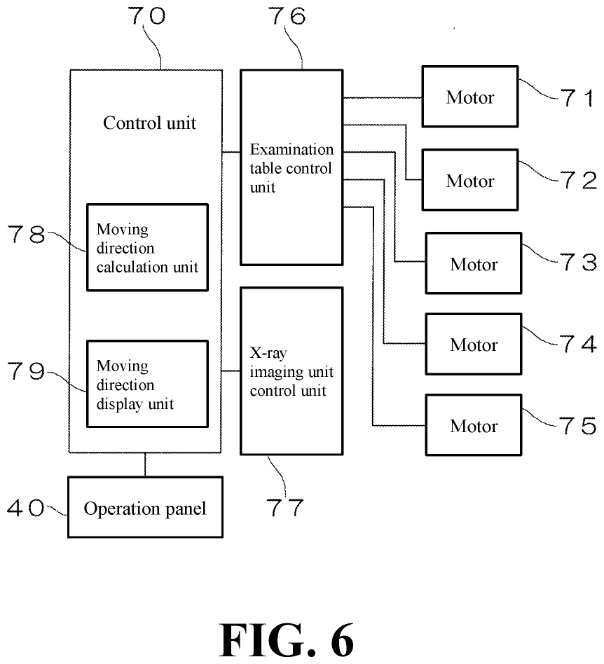

FIG. 6 is a block diagram showing the main control system of the above-described X-ray vascular fluoroscopic imaging apparatus.

This X-ray vascular fluoroscopic imaging apparatus is provided with a control unit 70 for controlling the entire apparatus. This control unit 70 is connected to the operation panel 40. The control unit 70 is provided with a moving direction calculation unit 78 for calculating the moving direction of the top board 11 when the top board 11 moves toward the final movement position from the current position of the top board 11 as a moving member and the selected final movement position, which will be described later. Further, the control unit 70 is provided with a moving direction display unit 79. The moving direction display unit 79 turns on the lamp built in the UP-button 54, the DOWN-button 55, the first button 62, the second button 64, the third button 63, or the fourth button 65 as the first operation portion corresponding to the moving direction of the top board 11 toward the final movement position calculated by the moving direction calculation unit 78 when the first position memory move button 56, the second position memory move button 57 or the origin returning button 61 as the second operation portion is operated, which will be described later.

This control unit 70 is connected to the examination table control unit 76 for controlling the examination table 1 and an X-ray imaging unit control unit 77 for controlling the X-ray imaging unit 2. The examination table control unit 76 is connected to a motor 71 for moving the top board 11 vertically, a motor 72 for moving the top board 11 in the longitudinal direction, a motor 73 for moving the top board 11 in the short direction, a motor 74 for rolling the top board 11, and a motor 75 for tilting the top board 11. These motors 71 72, 73, 74, and 75 are rotationally controlled by the control signal transmitted from the control unit 70 based on the operation of the operation panel 40.

Next, a moving operation for moving the top board 11 in the X-ray vascular fluoroscopic imaging apparatus having such a configuration will be described. Note that when moving the top board 11, either a manual operation in which an operator designates the moving direction (moving axis) of the top board 11 to move the top board 11 or an automatic operation in which an operator designates only the final movement position of the top board 11 to automatically move the top board 11 to the final movement position under the control of the control unit 70 is performed.

When manually moving the top board 11, an operator presses one of the UP-button 54, the DOWN-button 55, the first button 62, the second button 64, the third button 63, the fourth button 65 constituting the first operation portion. With this, the top board 11 moves in the direction corresponding to the button while the button is being pressed. For example, when raising the top board 11, an operator presses the UP-button 54. The top board 11 rises while the UP-button 54 is being pressed. When rolling the top board 11, an operator presses the roll/tilt selection button 66 and then presses the first button 62 or the second button 64. The top board 11 performs a rolling operation in the direction corresponding to the first button 62 or the second button 64 while the first button 62 or the second button 64 is being pressed.

When moving the top board 11 by such a manual operation, the top board 11 moves in the direction corresponding to the button pressed by the operator only while the operator is pressing the button. Therefore, since the operator sufficiently recognizes the movement of the top board 11 and the direction thereof, the operator does not collide with the moving top board 11.

On the other hand, when moving the top board 11 by the automatic operation, since the operator designates only the final movement position of the top board 11 and automatically moves the top board 11 to the final movement position by the control of the control unit 70, the operator may collide with the top board 11 because it is not known in which directions the top board 11 moves until the top board 11 starts moving. Therefore, in this X-ray vascular fluoroscopic imaging apparatus, a configuration is adopted in which the moving direction of the top board 11 is recognized by the operator by causing the button including any button of the UP-button 54, the DOWN-button 55, the first button 62, the second button 64, the third button 63, and the fourth button 65 to blink.

When moving the move top board 11 by an automatic operation, the operator presses one of the first position memory move button 56, the second position memory move button 57 and the origin returning button 61. Here, the first position memory move button 56 is pressed to automatically move the top board 11 to a preset first position as a final movement position. The second position memory move button 57 is pressed to automatically move the top board 11 to a preset second position as a final movement position. The origin returning button 61 is pressed to automatically move the top board 11 to the origin position as a final movement position.

When the first position memory move button 56, the second position memory move button 57, or the origin returning button 61 is pressed, the moving direction calculation unit 78 in the control unit 70 shown in FIG. 6 calculates the moving direction and the moving quantity of the top board 11 when the top board 11 is moved toward the final movement position based on the current position and the final movement position of the top board 11. Then, the moving direction display unit 79 in the control unit 70 shown in FIG. 6 causes the lamp included in a button corresponding to the moving direction of the top board 11 out of the UP-button 54, the DOWN-button 55, the first button 62, the second button 64, the third button 63, the fourth button 65, the long/short select button 69, the roll/tilt selection button 66 to blink. Thereafter, when the movement start button 67 is pressed, the top board 11 is moved toward the final movement position.

For example, in a state in which the position of the top board 11 currently facing the horizontal direction is 0 cm in the short direction and the longitudinal direction and 10 cm below the original point in the height direction, the moving direction display unit 79 will cause the lamp built in the UP-button 54 in the operation panel 40 to blink when the origin returning button 61 is pressed. As a result, the operator can recognize that the moving direction of the top board 11 is vertical. When the movement start button 67 is pressed, the top board 11 is raised by 10 cm by the driving of the motor 71 for moving the top board 11.

Also, for example, in a state in which the position of the top board 11 currently facing the horizontal direction is 0 cm from the original point in the short direction, is moved by 10 cm in the right direction from the original point in the longitudinal direction, and 0 cm from the original point in the height direction, the moving direction display unit 79 will cause the lamp built in the longitudinal/short selection button 69 and the lamp built in the fourth button 65 in the operation panel 40 to blink when the origin returning button 61 is pressed. As a result, the operator can recognize that the moving direction of the top board 11 is the longitudinal direction. When the movement start button 67 is pressed, the top board 11 is moved in the left direction by 10 cm by the driving of the motor 72 for moving the top board 11.

Further, for example, in a state in which the position of the top board 11 is 0 cm in the short direction and the longitudinal direction, 0 cm from the original point in the height direction, and is arranged at the rolled position, the moving direction display unit 79 will cause the lamp built in the roll/tilt selection button 66 and the lamp built in the button for tilting the top board 11 out of the first button 62 and the second button 64 in the operation panel 40 to blink when the origin returning button 61 is pressed. Thus, the operator can recognize that the moving direction of the top board 11 is the rolling direction of the board 11. When the movement start button 67 is pressed, the top board 11 performs a roll operation by the driving of the motor 74 for rolling the top board 11.

In all of the above-mentioned three specific examples, the case in which the origin returning button 61 is pressed has been described, but the same can be applied to the case in which the first position memory move button 56 or the second position memory move button 57 is pressed. Further, in the above-mentioned three specific examples, for convenience of explanation, the case in which the top board 11 moves only in one direction (one axial direction) has been explained, but when the top board 11 moves in a plurality of directions, the moving direction display unit 79 simultaneously causes a plurality of corresponding buttons out of the UP-button 54, the DOWN-button 55, the first button 62, the second button 64, the third button 63, the fourth button 65, the long/short select button 69, and the roll/tilt selection button 66 to blink.

As described above, in this X-ray vascular fluoroscopic imaging apparatus, when the first position memory move button 56, the second position memory move button 57 or the origin returning button 61 is pressed, the moving direction calculation unit 78 calculates the moving direction and the moving quantity of the top board 11 when moving the top board 11 toward the final movement position based on the current position and the final movement position of the top board 11, and the moving direction display unit 79 causes the lamp built in the button corresponding to the moving direction of the top board 11 out of the UP-button 54, the DOWN-button 55, the first button 62, the second button 64, the third button 63, the fourth button 65, the long/short select button 69, and the roll/tilt selection button 66 to blink. For this reason, after the operator recognizes the moving direction of the top board 11 in advance, the movement start button 67 can be pressed to initiate the movement of the top board 11.

In the embodiment described above, only the lamp built in one button corresponding to the moving direction of the top board 11 out of the UP-button 54 and the DOWN-button 55, the first button 62 and the second button 64, and the third button 63 and the fourth button 65, which are paired with each other with respect to the moving direction of the top board 11, is blinked, but both of buttons paired with each other may be blinked. Also, in this case, the operator can recognize in which direction the top board 11 moves.

Further, in the above-described embodiments, although the lamp built in a button is blinked in a blinking manner, it may be constantly turned on.

Further, in the above-described embodiments, although the respective buttons are composed of physically independent buttons, these buttons may be composed of buttons displayed on a touch panel type liquid crystal display unit.

Further, in the above-described embodiments, the case in which the examination table 1 constituting an X-ray fluoroscopic imaging apparatus is moved as a moving member according to the present invention by the operation panel 40 was described. However, when moving the C-shaped arm 28 by another operation panel, the present invention may be applied to the case in which the C-shaped arm 28, which is a support arm of the X-ray imaging unit 2 constituting an X-ray fluoroscopic imaging apparatus, is regarded as the moving member according to the present invention, in the same manner as described above.

### DESCRIPTION OF SYMBOLS

1: examination table
2: X-ray imaging unit
11: top board
12: flame
13: base
14: shaft
15: shaft
16: movable base
17: lifting base
18: support base
27: sliding portion
28: C-shaped arm
29: rotating portion
31: X-ray irradiation unit
32: flat panel detector
40: operation panel
70: control unit
71: motor
72: motor
73: motor
74: motor
75: motor
76: examination table control unit
77: X-ray imaging unit control unit
78: moving direction calculation unit
79: moving direction display unit

1. A radiographic imaging apparatus equipped with a moving member movable in a plurality of directions for performing radiographic imaging and an operation panel for operating movements of the moving member, the radiographic imaging apparatus comprising:
a plurality of first operation portions provided on the operation panel to move the moving member in a selected moving direction by being operated to select a moving direction of the moving member, the plurality of first operation portions being provided corresponding to moving directions of the moving member;
a second operation portion provided on the operation panel to prepare a movement of the moving member to a selected final movement position when the final movement position is selected by being operated;
a third operation portion arranged on the operation panel to initiate the movement of the moving member to the final movement position by being operated after the second operation portion is operated;
a lamp built in each of the plurality of first operation portions;
a moving direction calculation unit configured to calculate a moving direction of the moving member to the final movement position from a current position and the selected final movement position of the moving member; and
a moving direction display unit configured to turn on the lamp built in the first operation portion corresponding to the moving direction of the moving member to the final movement position calculated by the moving direction calculation unit when the second operation portion is operated.

2. The radiographic imaging apparatus as claimed in claim 1, wherein the moving direction display unit causes the lamp built in the first operation portion to blink.

3. The radiographic imaging apparatus as recited in claim claim 1,
wherein the moving member is a top board of an examination table movable in the plurality of directions with a subject placed thereon.

4. The radiographic imaging apparatus as recited in claim claim 1,
wherein the moving member is a support arm that supports a radiation irradiation unit and a radiation detection unit to move the radiation irradiation unit and the radiation detection unit in the plurality of directions.
